(19) United States
(12) Patent Application Publication
(10) Pub. No.: US 2010/0217369 A1
(43) Pub. Date: Aug. 26, 2010

(54) HAT FOR HAIR LOSS TREATMENT

(75) Inventor: Yossi GROSS, Moshav Mazor (IL)

Correspondence Address: SUGHRUE MION, PLLC, 2100 PENNSYLVANIA AVENUE, N.W., SUITE 800, WASHINGTON, DC, 20037, US

(73) Assignee: RAIBOW MEDICAL LTD., Herzliya (IL)

(21) Appl. No.: 12/683,815

(22) Filed: Jan. 7, 2010

Related U.S. Application Data
(60) Provisional application No. 61/204,574, filed on Jan. 7, 2009.

(51) Int. Cl. A61N 1/04

(52) U.S. Cl. 607/139

(57) ABSTRACT

Apparatus and methods are described for stimulating hair growth of a subject. A housing is placed on the subject's scalp, the housing having a plurality of at least 24 electrodes coupled thereto. A control unit drives the plurality of electrodes to stimulate hair growth by driving the plurality of electrodes to apply a current to the subject's scalp. Other embodiments are also described.

### CROSS-REFERENCES TO RELATED APPLICATIONS

The present application claims the benefit of U.S. Provisional Patent Application 61/204,574 to Gross, filed Jan. 7, 2009, entitled “Hat for hair loss treatment,” which is incorporated herein by reference.

### FIELD OF EMBODIMENTS OF THE INVENTION

The present invention generally relates to medical apparatus. Specifically, the present invention relates to an external medical apparatus for treating hair loss.

### BACKGROUND

Hair loss occurs most commonly on the head, but may affect any part of the body. Androgenetic alopecia is the most common type of hair loss, affecting about half of all men and 10 to 20% of women. Hair loss may develop gradually or suddenly. It results from hereditary factors, aging, local skin conditions, and diseases that affect the body generally (i.e., systemic diseases). Many drugs can also cause hair loss.

Minoxidil, known under the brand name Rogaine®, is a drug approved by the FDA for treating hair loss. Rogaine® is applied directly to scalp skin where cessation of hair loss and stimulation of new hair growth is desired. Rogaine® is described as working by revitalizing shrunken hair follicles, thus increasing their size. When applied daily, hair follicles are described as increasing in size, and the growing phase becoming longer, producing longer, thicker hairs. Minoxidil's ability to stimulate hair growth and treat hair loss may be due to it having nitric oxide-related vasodilatory properties.

U.S. Pat. No. 6,332,097 to Beder et al. describes a plurality of electrodes mounted within a hood positionable over a subject's head to form an array of concentric electrically conductive electrode rings. Beder et al. describe how the rings can be applied to an outer surface of an electrically insulating liner insertable within the hood. The array is described as preferably comprising five rings mounted within the hood to subtend a 90 degree arc on both sides of a central perpendicular axis of the hood. The four upper electrodes each subtend an arc of about 15 degrees, and the fifth (lower) electrode subtends an arc of about 6 degrees, with 6 degree arc gaps between each pair of electrodes on either side of the central perpendicular axis. A voltage pulse generator's output signal is described as being connected across the two upper electrode pairs, with alternate electrodes being connected to one of the two output terminals of the voltage pulse generator. A voltage divider applies a reduced voltage signal to the lower electrode.

U.S. Pat. No. 5,344,440 to Stephen describes an improved method and apparatus for stimulating the growth and healing of living, especially human tissues, promoting the healing of wounds and skeletal fractures. Stephen describes an electrical apparatus that consists of a baseplate, made of lightweight nonconductive material, for encompassing all or a portion of a body member. A multiplicity of generally evenly spaced holes are provided in the baseplate and a probe adapter including a moveable electrically conductive probe is situated within the holes. An electric contact connects the interior of each adapter and the probe situated therein to a terminal on the baseplate. Each probe is described as being individually adjustable to contact the wearer's body part within the base member, regardless of the configuration of the body part, thus allowing treatment of all or a selected portion of the body part.

U.S. Pat. No. 6,834,206 to Pitzen et al. describes a method for applying a therapeutic signal to a body portion of a subject to encourage hair growth. The apparatus is described as including a first electrode and a second electrode. The first electrode is secured in contact with the skin surface of the subject remote from the body portion, and the second electrode is positioned at the body portion. The therapeutic signal is provided at a conductive pod of the second electrode. The second electrode is manipulated by an operator to apply a circular motion to the body portion underlying the conductive pod. The circular motion is described as being repetitively applied as the conductive pod is moved across the body portion to loosen connective tissue at the body portion.

U.S. Pat. No. 5,800,477 to Groux describes a method and apparatus for growing hair on a subject's scalp. The method includes pinching an area of a subject's scalp having hair bulbs for at least 2 seconds with a pair of electrodes. The electrodes are energized so that they pass a low voltage, low current and low frequency signal through the pinched area of the subject's scalp. The frequency of the signal is described as being preferably less than about 6 Hz and the current thereof is preferably between about 50 and 800 microamps. The Groux patent describes how the foregoing process is periodically repeated over several weeks, preferably months, to regrow or enhance the growth of hair on the treated area. The frequency and current of the signal are also described as being varied in each treatment for optimum results.

The following patents and patent applications may be of interest:

U.S. Pat. No. 7,229,403 to Schock et al.

U.S. Pat. No. 7,206,637 to Salo

U.S. Pat. No. 6,939,345 to KenKnight et al.

U.S. Pat. No. 6,896,651 to Gross et al.

U.S. Pat. No. 6,871,092 to Piccone

U.S. Pat. No. 6,865,416 to Dev et al.

U.S. Pat. No. 6,862,480 to Cohen et al.

U.S. Pat. No. 6,845,267 to Harrison et al.

U.S. Pat. No. 6,824,561 to Soykan et al.

U.S. Pat. No. 6,810,286 to Donovan et al.

U.S. Pat. No. 6,741,895 to Gafni et al.

U.S. Pat. No. 6,485,524 to Strecker

U.S. Pat. No. 6,479,045 to Bologna et al.

U.S. Pat. No. 6,463,323 to Conrad-Vlasak et al.

U.S. Pat. No. 6,432,037 to Eini et al.

U.S. Pat. No. 6,347,247 to Dev et al.

U.S. Pat. No. 6,245,103 to Stinson

U.S. Pat. No. 6,200,259 to March

U.S. Pat. No. 6,139,538 to Houghton et al.

U.S. Pat. No. 6,086,527 to Talpade

U.S. Pat. No. 6,058,331 to King

U.S. Pat. No. 6,030,375 to Anderson et al.

U.S. Pat. No. 5,906,641 to Thompson et al.

U.S. Pat. No. 5,800,502 to Boutos

U.S. Pat. No. 5,800,501 to Sherlock et al.

U.S. Pat. No. 5,324,323 to Bui

U.S. Pat. No. 5,251,623 to Groux et al.

U.S. Pat. No. 5,046,511 to Mauer et al.

U.S. Pat. No. 5,830,848 to Harrison et al.

U.S. Pat. No. 4,827,946 to Kaali et al.

U.S. Design Pat. No. 415,835 to Malewicz et al.

U.S. Pat. No. 3,872,859 to Pitzen et al.

US Patent Application Publication 1998/34677 to Hilburg et al.

US Patent Application Publication 2002/0010414 to Coston

US Patent Application Publication 2002/0103454 to Sackner et al.

US Patent Application Publication 2003/0036773 to Whitehurst et al.

US Patent Application Publication 2003/0204206 to Padua et al.

US Patent Application Publication 2003/057264 to Geiser et al.

US Patent Application Publication 2004/0039417 to Soykan et al.

US Patent Application Publication 2005/0119605 to Sohn

US Patent Application Publication 2005/0038473 to Tamarkin et al.

US Patent Application Publication 2006/0276844 to Alon et al.

US Patent Application Publication 2007/0196428 to Glauser et al.

US Patent Application Publication 2007/0248676 to Stamler et al.

PCT Publication WO 00/002501 to Benjamin et al.

PCT Publication WO 03/00314 to Sohn

PCT Publication WO 04/014456 to Allen et al.

PCT Publication WO 06/094273 to White et al.

PCT Publication WO 07/106533 to Stern et al.

PCT Publication WO 07/113833 to Cahan et al.

PCT Publication WO 2006/064503 to Belsky et al.

PCT Publication WO 2006/123346 to Alon et al.

European Patent Application Publication EP 0 109 935 A1 to Charmillot et al.

### SUMMARY OF EMBODIMENTS OF THE INVENTION

For some applications of the present invention, a plurality of typically at least 24, for example, 24-100, electrodes are coupled to a housing configured to be placed on the scalp of a subject. A control unit is configured to stimulate hair growth by individually addressing subsets of one or more of the plurality of electrodes to apply a current into the scalp of the subject. Typically, the current is configured to induce nitric oxide production, which in turn enhances hair growth.

The inventor has identified that when there is a large scalp area through which current can pass, the current does not uniformly pass through the area, but instead travels along one or more smaller paths of least resistance. By contrast, in these applications of the present invention, due to the alignment of the electrodes in the housing, the current is applied in many discrete, small regions, rather than across broad regions of the scalp. Application of the current in many discrete regions provides a more uniform distribution of the current, and therefore a more uniform pattern of hair regrowth.

For some applications, the plurality of electrodes comprises a first and a second subset of two or more electrodes. In such an application, the control unit is configured to individually address the first and the second subsets of electrodes, by driving the current into the first and the second subsets of electrodes, in sequence, to apply the current to the scalp of the subject.

In another application, the plurality of electrodes comprises a first, a second, a third and a fourth subset of two or more electrodes. In such an application, the control unit is configured to individually address the subsets, by driving the current into the subsets of electrodes in sequence, to apply the current to the scalp.

Use of multiple subsets typically provides a more uniform distribution of current, and a more uniform pattern of hair regrowth.

There is therefore provided, in accordance with some applications of the present invention, apparatus for stimulating hair growth of a subject, including:

a housing configured for placement on a scalp of the subject;

a plurality of at least 24 electrodes coupled to the housing; and a control unit configured to drive the plurality of electrodes to stimulate hair growth by driving the plurality of electrodes to apply a current to the scalp of the subject.

For some applications, the plurality of electrodes includes 24 to 100 electrodes.

For some applications, the control unit is configured to enhance nitric oxide production by tissue of the subject by driving the plurality of electrodes to apply the current.

For some applications, at least 75% of the plurality of electrodes have a scalp-contact surface area of less than 10 mm2.

For some applications, the plurality of electrodes are arranged in an array formation.

For some applications, the control unit is configured to individually address subsets of two or more of the plurality of electrodes.

For some applications, the control unit sets the current to have an amplitude that is between 100 uA and 4 mA.

For some applications, the plurality of electrodes includes a first subset of two or more of the electrodes and a second subset of two or more of the electrodes, and the control unit is configured to individually address the first and second subsets by driving the first and second subsets of the electrodes, in sequence, to apply the current to the scalp.

For some applications, the plurality of electrodes includes a first subset of two or more of the electrodes, a second subset of two or more of the electrodes, a third subset of two or more of the electrodes, and a fourth subset of two or more of the electrodes, and the control unit is configured to individually address the subsets by driving the subsets, in sequence, to apply the current to the scalp.

For some applications, the plurality of electrodes includes a first and a second electrode, and the control unit is configured to:

apply a first pulse of current to the scalp via the first electrode,

subsequently, apply a second pulse of current to the scalp via the second electrode, and

subsequently, apply a third pulse of current to the scalp via the first electrode,

without applying a pulse of current to the scalp via the first electrode, between applying the first and third pulses.

For some applications, the plurality of electrodes includes a first and a second electrode, and the control unit is configured to:

apply a first pulse of current to the scalp via the first electrode,

subsequently, apply a second pulse of current to the scalp via the first electrode, and

subsequently, apply a third pulse of current to the scalp via the second electrode,

without applying a pulse of current to the scalp via the second electrode, between applying the first and second pulses.

For some applications, the control unit configures the current to have a frequency applied to at least one of the electrodes that is between 6 Hz and 20 Hz.

For some applications, the control unit is configured to activate the plurality of electrodes in repeated waves of activation, in which at least two of the electrodes apply current to the scalp non-simultaneously, each of the electrodes applying its respective current at a respective frequency between 6 Hz and 20 Hz.

For some applications, at least 75% of the plurality of electrodes are within 5 mm of another one of the electrodes.

For some applications, at least 75% of the plurality of electrodes are within 1 mm of another one of the electrodes.

There is additionally provided, in accordance with some applications of the present invention, a method for stimulating hair growth of a subject, including:

placing a plurality of at least 24 electrodes on a scalp of the subject; and

via the electrodes, driving into the scalp of the subject an electric current configured to stimulate hair growth.

The present invention will be more fully understood from the following detailed description of embodiments thereof, taken together with the drawing, in which:

### BRIEF DESCRIPTION OF THE DRAWING

FIG. 1 is a schematic illustration of a device for treating hair loss, in accordance with some applications of the present invention.

### DETAILED DESCRIPTION OF EMBODIMENTS

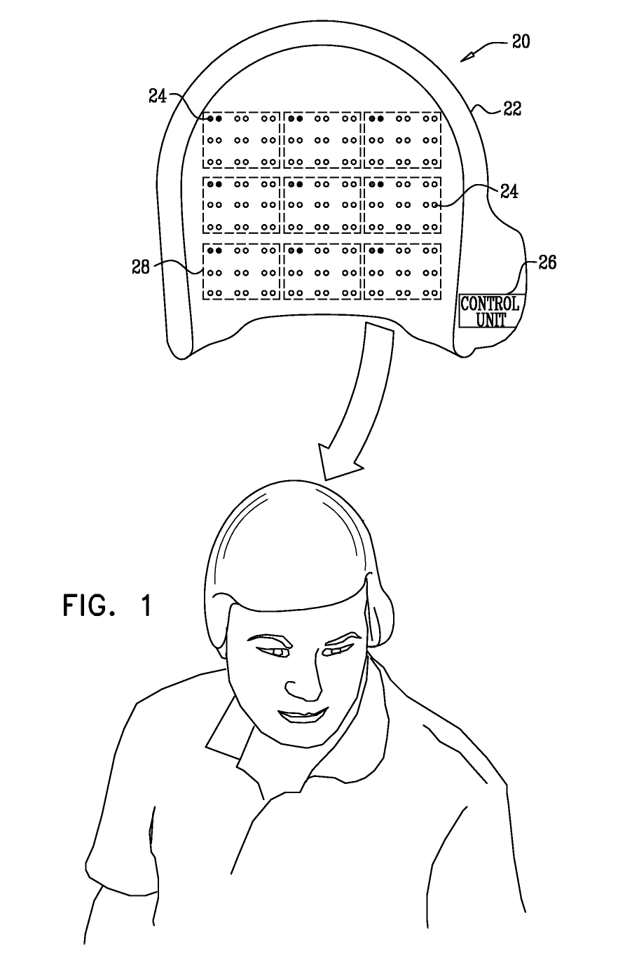

Reference is now made to FIG. 1, which is a schematic illustration of a hair loss treatment device 20, in accordance with some applications of the present invention. Hair loss treatment device 20 comprises a plurality of at least 24, for example, 24-100, electrodes 24, coupled to a housing 22, such as a hat that fits securely to the head of a subject. A control unit 26 is configured to drive electrodes 24 to apply an electric current into the scalp of the subject configured to stimulate hair growth, typically by inducing nitric oxide production. Typically, control unit 26 is configured to individually address subsets of two or more of the plurality of electrodes 24, to drive the subsets to apply the current into the scalp of the subject.

Typically, at least 75% of electrodes 24 have a scalp-contact surface area of 0.2 mm2 to 15 mm2, or, less than 10 mm2. Typically, the scalp-contact surface of each of the electrodes is rounded. Typically, the plurality of electrodes are arranged in an array, such that at least 75% of the plurality of electrodes are within 5 mm, for example, within 1 mm, of another one of the electrodes. For some applications, at least 75% of the plurality of electrodes are within less than 1 mm of another one of the electrodes.

Control unit 26 typically stimulates hair growth in the scalp of the subject by driving through each electrode an electric current with an amplitude between 100 uA and 4 mA, for example, about 1 mA, into a portion of the scalp of the subject. Typically, the electric current has a frequency between 6 Hz and 20 Hz.

For some applications, a pulse (e.g., a pulse lasting 0.5 ms to 3 ms) is applied to the scalp via a first electrode pair, or a first electrode. Subsequently, a pulse is applied to the scalp via a second electrode pair, or single electrode, and so on. For some applications, having applied a current to the scalp via all of the electrodes, the cycle of applying the current to the scalp via respective electrode pairs (or electrodes) is then repeated. A time interval between the initiation of the first cycle and the initiation of the second cycle is typically about 10-200 ms, e.g., 15-45 ms (e.g., 30 ms) or 45-200 ms. In this manner, for any given electrode, the frequency of application can be a desired value, such as 6-20 Hz. For some applications, a 6-20 Hz train of pulses is applied to the scalp via a first electrode pair, or electrode, or set of electrodes. Subsequently, a train of pulses is applied to the scalp via a second electrode pair, or electrode, or set of electrodes.

For some applications, device 20 comprises a first subset and a second subset of two or more electrodes 24 coupled to housing 22. Control unit 26 is configured to individually address the first and the second subsets of electrodes 24, by driving the first and the second subsets of electrodes 24, in sequence, to apply the current to the scalp of the subject. The darkened pairs of electrodes in FIG. 1 are an example of a first subset of electrodes, and in this embodiment are driven at the same time. The non-darkened pairs immediately to the right of the darkened pairs are an example of a second subset of electrodes 24, and these non-darkened pairs are driven at the same time, but after the driving of the darkened pairs. Electrodes 24 in each subset apply a localized current to a local portion of the scalp. These localized currents generally travel along the path of least resistance through the scalp of the subject, between two electrodes, thereby stimulating hair growth. Typically, a large number of subsets of the electrodes are individually addressable, so as to provide current application to a large number of discrete regions of the scalp.

For some applications, electrodes 24 are divided into a plurality of sub-arrays 28 of electrodes 24. FIG.

1, for example, shows nine sub-arrays 28, each sub-array containing nine pairs of electrodes 24. Typically, one pair of electrodes 24 in each sub-array 28 is individually addressed by control unit 26 at any given time, so as to provide current application to a large number of discrete regions of the scalp at the same time. Subsequently, control unit 26 addresses another pair of electrodes 24 of each of the sub-arrays, in order to drive current into the portion of the scalp covered by the other pair. In sequence, the control unit then applies current into the scalp under each of the pairs of electrodes. (The pairs of electrodes that are addressed at any given time constitute a subset, as described above.)

In another embodiment, the electrodes are not divided into sub-arrays, and the manner in which control unit addresses the electrodes is not related to an arrangement of sub-arrays of electrodes. Typically, in this case, the control unit causes a wave of activation by addressing each of the electrodes (or electrode pairs) in sequence, such that the current applied by a given electrode to the scalp has a desired frequency, such as 6-20 Hz.

For some applications, only a portion of the scalp of the subject requires the use of hair loss treatment device 20. In such an application, control unit 26 is configured to individually address only a subsection of electrodes 24 positioned local to this portion of the scalp of the subject. Alternatively or additionally, the electrodes are disposed within device 20 so as to reflect common hair loss patterns (e.g., male pattern baldness). Further alternatively or additionally, device 20 is removable from a hat and securable at a different site in the hat in order to allow treatment of a different portion of the scalp.

It will be appreciated by persons skilled in the art that the present invention is not limited to what has been particularly shown and described hereinabove.

Rather, the scope of the present invention includes both combinations and subcombinations of the various features described hereinabove, as well as variations and modifications thereof that are not in the prior art, which would occur to persons skilled in the art upon reading the foregoing description.

1. Apparatus for stimulating hair growth of a subject, comprising:
a housing configured for placement on a scalp of the subject;
a plurality of at least 24 electrodes coupled to the housing; and
a control unit configured to drive the plurality of electrodes to stimulate hair growth by driving the plurality of electrodes to apply a current to the scalp of the subject.

2. The apparatus according to claim 1, wherein the plurality of electrodes comprises 24 to 100 electrodes.

3. The apparatus according to claim 1, wherein the control unit is configured to enhance nitric oxide production by tissue of the subject by driving the plurality of electrodes to apply the current.

4. The apparatus according to claim 1, wherein at least 75% of the plurality of electrodes have a scalp-contact surface area of less than 10 mm2.

5. The apparatus according to claim 1, wherein the plurality of electrodes are arranged in an array formation.

6. The apparatus according to claim 1, wherein the control unit is configured to individually address subsets of two or more of the plurality of electrodes.

7. The apparatus according to claim 1, wherein the control unit sets the current to have an amplitude that is between 100 uA and 4 mA.

8. The apparatus according to claim 1, wherein the plurality of electrodes comprises a first subset of two or more of the electrodes and a second subset of two or more of the electrodes, and wherein the control unit is configured to individually address the first and second subsets by driving the first and second subsets of the electrodes, in sequence, to apply the current to the scalp.

9. The apparatus according to claim 1, wherein the plurality of electrodes comprises a first subset of two or more of the electrodes, a second subset of two or more of the electrodes, a third subset of two or more of the electrodes, and a fourth subset of two or more of the electrodes, and wherein the control unit is configured to individually address the subsets by driving the subsets, in sequence, to apply the current to the scalp.

10. The apparatus according to claim 1, wherein the plurality of electrodes includes a first and a second electrode, and wherein the control unit is configured to:
apply a first pulse of current to the scalp via the first electrode,
subsequently, apply a second pulse of current to the scalp via the second electrode, and
subsequently, apply a third pulse of current to the scalp via the first electrode,
without applying a pulse of current to the scalp via the first electrode, between applying the first and third pulses.

11. The apparatus according to claim 1, wherein the plurality of electrodes includes a first and a second electrode, and wherein the control unit is configured to:
apply a first pulse of current to the scalp via the first electrode,
subsequently, apply a second pulse of current to the scalp via the first electrode, and
subsequently, apply a third pulse of current to the scalp via the second electrode,
without applying a pulse of current to the scalp via the second electrode, between applying the first and second pulses.

12. The apparatus according to claim 1, wherein the control unit configures the current to have a frequency applied to at least one of the electrodes that is between 6 Hz and 20 Hz.

13. The apparatus according to claim 12, wherein the control unit is configured to activate the plurality of electrodes in repeated waves of activation, in which at least two of the electrodes apply current to the scalp non-simultaneously, each of the electrodes applying its respective current at a respective frequency between 6 Hz and 20 Hz.

14. The apparatus according to claim 1, wherein at least 75% of the plurality of electrodes are within 5 mm of another one of the electrodes.

15. The apparatus according to claim 14, wherein at least 75% of the plurality of electrodes are within 1 mm of another one of the electrodes.

16. A method for stimulating hair growth of a subject, comprising:
placing a plurality of at least 24 electrodes on a scalp of the subject; and
via the electrodes, driving into the scalp of the subject an electric current configured to stimulate hair growth.

17. The method according to claim 16, wherein placing the plurality of electrodes on the scalp comprises placing 24-100 electrodes on the scalp.

18. The method according to claim 16, wherein driving the electric current comprises enhancing nitric oxide production by tissue of the subject.

19. The method according to claim 16, wherein driving the electric current comprises configuring the electric current to have an amplitude between 100 uA and 4 mA.

20. The method according to claim 16, wherein placing the plurality of electrodes on the scalp of the subject comprises arranging the electrodes in an array formation.

21. The method according to claim 16, wherein driving the electric current comprises driving the electric current in a sequence through a plurality of individually addressable subsets of the electrodes.

22. The method according to claim 16, wherein driving the electric current comprises driving the electric current, in a sequence, through a first subset of two or more of the electrodes, a second subset of two or more of the electrodes, a third subset of two or more of the electrodes, and a fourth subset of two or more of the electrodes.

23. The method according to claim 16, wherein driving the electric current comprises:
driving a first pulse of current into the scalp via a first electrode,
subsequently, driving a second pulse of current into the scalp via a second electrode, and
subsequently, driving a third pulse of current into the scalp via the first electrode,
without driving a pulse of current into the scalp via the first electrode, between driving the first and third pulses.

24. The method according to claim 16, wherein driving the electric current comprises:
driving a first pulse of current into the scalp via a first electrode,
subsequently, driving a second pulse of current into the scalp via the first electrode, and
subsequently, driving a third pulse of current into the scalp via a second electrode,
without driving a pulse of current into the scalp via the second electrode, between driving the first and second pulses.

25. The method according to claim 16, wherein driving the current comprises configuring the current to have a frequency applied to at least one of the electrodes that is between 6 Hz and 20 Hz.

26. The method according to claim 25, wherein driving the current comprises activating the plurality of electrodes in repeated waves of activation, in which at least two of the electrodes apply current to the scalp non-simultaneously, each of the electrodes applying its respective current at a respective frequency between 6 Hz and 20 Hz.

27. The method according to claim 16, wherein placing the plurality of electrodes comprises placing at least 75% of the plurality of electrodes within 5 mm of another one of the electrodes.

28. The method according to claim 27, wherein placing the plurality of electrodes comprises placing at least 75% of the plurality of electrodes within 1 mm of another one of the electrodes.
